(19) United States
(12) Patent Application Publication
(10) Pub. No.: US 2005/0043560 A1
(43) Pub. Date: Feb. 24, 2005

(54) Novel beta-hydroxyamides

(75) Inventors: Mircea Manea, Vittsjo (SE); Cecilia Petersson, Helsingborg (SE)

Correspondence Address: STEVENS DAVIS MILLER & MOSHER, LLP, 1615 L STREET, NW, SUITE 850, WASHINGTON, DC, 20036, US

(73) Assignee: Pergo (Europe) AB, Trelleborg (SE)

(21) Appl. No.: 10/919,483

(22) Filed: Aug. 17, 2004

Related U.S. Application Data
(63) Continuation-in-part of application No. 10/311,295, filed on Feb. 4, 2003, now abandoned.

(30) Foreign Application Priority Data
Jun. 19, 2000 (SE) 0002268-1

(51) Int. Cl. C07C 233/02

(52) U.S. Cl. 560/155

(57) ABSTRACT

Disclosed is a novel β-hydroxylamide of general formula 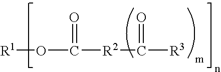
wherein R¹ is a linear or branched aliphatic hydrocarbon group optionally comprising one or more ether and/or ester units, such as a group derived from at least one hydroxyfunctional
compound, R² is a linear or branched aliphatic and/or aromatic hydrocarbon group, such as a group derived from at least one carboxyfunctional compound or at least one anhydride halide or ester of a carboxyfunctional compound, R³ is N-alkyl or N-cycloalkyl for instance derived from at least one alkanolamine, and wherein m is an integer and at least 1 and n is an integer and at least 2, In a further aspect the present invention refers to a process for synthesis of said β-hydroxyamide. The process comprises the Steps of (i) subjecting a di, tri or polyalcohol to alcoholysis with at least one di, tri or polyalkyl ester of a di, tri or polyfunctional carboxylic acid and (ii) subjecting obtained reaction product to aminolysis with at least one alkanolamine.

### CROSS REFERENCE TO RELATED APPLICATIONS

This application is a Continuation-in-Part of U.S. application Ser. No. 10/311,295, filed Feb. 4, 2003, which in turn claims priority from PCT International Application No. PCT/SE01/01359, filed Dec. 17, 2002, which in turn claims priority from Swedish Application No. 0002268-1, filed Jun. 19, 2000, each of which is incorporated by reference in its entirety.

### BACKGROUND OF THE INVENTION

The present invention refers to a novel series of β-hydroxyamides, such as β-hydroxyalkylamides, β-hydroxyarylalkylamides and β-hydroxyalkylarylamides, having a core being derived from a di, tri or polyhydric compound, to which core at least one alkylamide, arylalkylamide or alkylarylamide core branch is bonded. Said P-hydroxyamides are useful as chemical intermediates and chemical crosslinkers and/or curing agents. In a further aspect, the present invention refers to a process for synthesis of said β-hydroxyamides, which process comprises the Steps of (i) alcoholysis of said di, tri or polyhydric compound and a di, tri or poly(alkyl) ester and (ii) aminolysis of obtained reaction product with an alkanolamine.

β-hydroxyamides are well known in coating applications such as powder coatings as a challenging alternative to compounds such as triglycidyl trisisocyanurate (TGIC). β-hydroxyamides are normally produced by aminolysis of alkyl esters, such as diethyl esters of dicarboxylic acids, by β-aminoalcohols. β-hydroxyamides are normally solid and used for instance in powder coating compositions as crosslinkers and/or curing agents. Available patent literature discloses a number of processes for production of P-hydroxyamides, p-hydroxyamides yielded in said processes and various application areas for said P-hydroxyamides. European Patent Application 0 473 380, European Patent Application 0 960 878, U.S. Pat. No. 4,076,917, U.S. Pat. No. 4,727,111 and U.S. Pat. No. 5,101,073 disclose β-hydroxyalkylamides and processes for production of β-hydroxyalkylamides. Disclosed products are reaction products of a di, tri or polyfunctional carboxylic acid and at least one β-aminoalcohol. U.S. Pat. No. 2,703,798 and International Patent Applications WO 92/06070, WO 92/06072 and WO 92/06073 disclose processes yielding reaction products of aliphatic fatty acids and N-alkylglucamines. German Patent Applications 31 50 269 and 31 24 885 relate to poly-N,N-hydroxyalkylamides of di, tri or polyfunctional aromatic or cycloaliphatic carboxylic acids. Each of the references discussed in this paragraph is hereby incorporated by reference in its entirety.

In spite of a strong expansion in powder coatings, there are still segments wherein the use of liquid coatings, presently for instance comprising saturated polyesters or alkyds combined with alkyl etherified urea-formaldehyde and/or melamine-formaldehyde resins as crosslinkers, is required. Various coating systems, binders, solvents and crosslinkers are thoroughly disclosed and discussed in readily available handbooks, such as “A Manual for Resins for Surface Coatings” Vol. I-III, G. Hayward, P. K. T. Oldring and C. J. S. Standen, SITA Technology, London, 1993-94, “Surface Coatings—Science & Technology”, S. Paul (ed.), John Wiley & Sons, 1996 and “Surface Coatings” vol. 1, “Raw Materials and Their Usage” and vol. 2, “Paints and Their Application”, Chapman & Hall Ltd, London, 1974 and 1984 (each of which is hereby incorporated by reference in its entirety). Environmental issues regarding for instance the minimizing of organic solvents and formaldehyde emission reduce the formulation possibilities when high performance is required or requested. The coil coating sector and other application areas wherein waterborne systems do not perform as well as solventborne systems, and where powder coatings are not performing adequately require the presence of a suitable crosslinker complying with environmental issues as well as demands on high performance. Each of the references discussed in this paragraph is hereby incorporated by reference in its entirety.

### SUMMARY OF THE INVENTION

It has quite unexpectedly been found that β-hydroxylamides having a core derived from a di, tri or polyfunctional alcohol give the following advantages over presently known and available crosslinkers, including β-hydroxyamides being the reaction products of di, tri or polyfunctional carboxylic acids and aminoalcohols, coatings can be formulated as a solventborne or waterborne system, solventborne coatings can be formulated as high-solids systems, crosslinking temperature may be moderate to high in the range of 150-200° C., compatibility problems are due to the di, tri or polyhydric core compound reduced, the relation between flexibility and hardness is improved compared to formulations comprising amino-formaldehyde resins as crosslinkers, and the crosslinking density can be tailored by a proper selection of the di, tri or polyhydric core compound.

### DETAILED DESCRIPTION

The β-hydroxyamide of the present invention is a compound of general formula 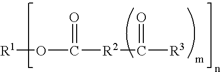
wherein:
substituent R¹ can be a linear, branched or cyclic, saturated or unsaturated aliphatic hydrocarbon group optionally comprising one or more, such as at least two, ether and/or ester units, which substituent in preferred embodiments is derived from at least one compound having at least two hydroxyl groups. R¹ is in a preferred embodiment a group of formula —CₙH₂ₙ—;
substituent R² is a linear, branched or cyclic, saturated or unsaturated aliphatic and/or aromatic hydrocarbon group, which substituent in preferred embodiments is derived from at least one compound having at least two carboxyl groups or from an anhydride, a halide or an alkyl ester or ether of a compound having said at least two carboxyl groups. In a preferred embodiment, R² can be an alkylene group, i.e., a bivalent radical derived from an aliphatic hydrocarbon by removing two hydrogen atoms from different terminal carbons, only if, however, the hydrocarbon is of the formula CH₄, both hydrogens are removed from the sole carbon (See IUPAC Nomenclature of Organic Chemistry: Rule A-4.3) or an arylene group, i.e., a bivalent aromatic hydrocarbon radical (See IUPAC Nomenclature of Organic Chemistry: Rules 11.6 and 13.5).
substituent R³ is N-alkyl or N-cycloalkyl having at least one hydroxyl group in β-position, which substituent in preferred embodiments is derived from at least one alkanolamine,
m is an integer and at least 1, and
n is an integer and at least 2.

Substituent R³ is in preferred embodiments a group of formula 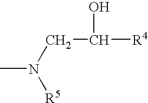
wherein R⁴ is alkyl and R⁵ is hydrogen or a group of formula 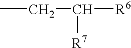
wherein R⁶ is alkyl and R⁷ is hydrogen, hydroxyl, alkyl or hydroxyalkyl, wherein alkyl is alkanyl having 1-24 carbon atoms or alkenyl having 2-24 carbon atoms.

The β-hydroxyamide of the present invention include embodiments wherein:
said R¹ is a saturated hydrocarbon group having 1-24 carbon atoms or an unsaturated hydrocarbon group having 2-24 carbon atoms,
said R² is a saturated hydrocarbon group having 1-24 carbon atoms or an unsaturated hydrocarbon group having 2-24 carbon atoms,
said N-cycloalkyl is N-cycloalkanyl having 3-20 carbon atoms, and
said one or more ether units is/are derived from at least one alkylene oxide, such as ethylene oxide, propylene oxide and/or butylene oxide.

The compound having said at least two hydroxyl groups is in preferred embodiments of the present invention a di, tri or polyalcohol of formula 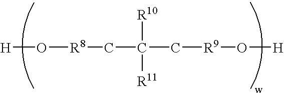
wherein
w is an integer and at least 1,
R⁸ and R⁹ each independently is a group of formula —(CₓH₂ₓ)_{y}—, —(CᵣH₂ᵣOₚ₋₁)ₚ— or —(CₓH₂ₓ)_{y}(CᵣH₂ᵣOₚ₋₁)ₚ—, and
R¹⁰ and R¹¹ each independently is —H, —OH, —COOH or a group of formula —(CₓH₂ₓ₊₁)_{y}, —(CₓH₂ₓ)_{y}OH, —(CₓH₂ₓ)_{y}COOH, —(CᵣH₂ᵣO)ₚH, —(CₓH₂ₓ)_{y}(CᵣH₂ᵣO)ₚH, wherein x, y, r and p are independent integers being at least 1.

Substituent R² is preferably derived from a di, tri or polyfunctional carboxylic acid, from an anhydride of a di, tri or polyfunctional carboxylic acid or from a di, tri or polyalkyl ester of a di, tri or polyfunctional carboxylic acid. Said carboxylic acid is preferably linear or branched having 1-18, such as 1-8 or 1-4, carbon atoms.

The compound having said at least two hydroxyl groups is advantageously a di, tri or polyalcohol selected from the group consisting of a 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol. Alkyl in these embodiments is preferably linear or branched alkanyl having 1-18 carbon atoms or linear or branched alkenyl having 3-18 carbon atoms and alkoxy and hydroxyalkoxy is for example derived from at least one alkylene oxide, such as ethylene oxide, propylene oxide and/or butylene oxide. The di, tri or polyalcohol can suitably be exemplified by compounds such as 2-methyl-1,3-propanediol, 2-methyl-2-ethyl-1,3-propanediol, 2-ethyl-2-butyl-1,3-propanediol, neopentyl glycol, dimethylolpropane, trimethylolethane, trimethylolpropane, di(trimethylolethane), di(trimethylolpropane), pentaerythritol and dipentaerythritol.

Further suitable embodiments of the compound having said at least two hydroxyl groups are found among alcohols such as glycerol, diglycerol, anhydroenneaheptitol, sorbitol and mannitol as well as monoallyl or mono(methallyl) ethers of glycerol, trimethylolethane, trimethylolpropane, di(trimethylolethane), di(trimethylolpropane) and pentaerythritol and diallyl or di(methallyl) ethers of di(trimethylolethane), di(trimethylolpropane) or pentaerythritol.

Yet further suitable embodiments of the compound having said at least two hydroxyl groups include di, tri or poly(hydroxy)carboxylic acids, wherein the carboxyl group or groups optionally is/are protected using for instance well known protection methods as disclosed in handbooks such as “Protective Groups in Organic Synthesis” chapter 5 “Protection for the Carboxyl Group” by Theodora W. Green and Peter G. M. Wuts, John Wiley & Sons Inc., 1991 (hereby incorporated by reference in its entirety). Said hydroxycarboxylic acids are most preferably selected from the group consisting of 2,2-dimethylolpropionic acid, α,αbis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)malonic acid and gluconic acid.

The compound having said at least two carboxyl groups is most preferably a di, tri or polyfunctional carboxylic acid, which in the most preferred embodiments is selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid, or is an anhydride, a halide or an alkyl ester or ether of a said di, tri or polyfunctional carboxylic acid.

The alkanolamine as disclosed above is advantageously selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclohexanol, 2-aminocyclopentanol, 2-amino-2-methyl-1,3-propanediol, 2-amino-2-ethyl-1,3-propanediol and 2-amino-2-hydroxymethyl-1,3-propanediol.

In a further aspect, the present invention refers to a process for synthesis of a p-hydroxyamide as disclosed above. The process comprises preferably the steps of (i) subjecting a di, tri or polyalcohol of formula 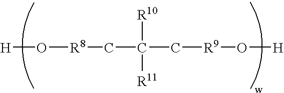
wherein
   w is an integer and at least 1, R⁸ and R⁹ each independently is a group of formula —(CₓH₂ₓ)_{y}—, —(CᵣH₂ᵣOₚ₋₁)ₚ— or —(CₓH₂ₓ)_{y}(CᵣH₂ᵣOₚ₋₁)ₚ—, and
   R¹⁰ and R¹¹ each independently is —H, —OH, —COOH or a group of formula —(CₓH₂ₓ₊₁)_{y}, —(CₓH₂ₓ)_{y}OH, —(CₓH₂ₓ)_{y}COOH, —CᵣH₂ᵣO)ₚH, —(CₓH₂ₓ)_{y}(CᵣH₂ᵣO)ₚH, wherein x, y, r and p are independent integers being at least 1,
to alcoholysis with a di, tri or polyalkyl ester of a di, tri or polyfunctional carboxylic acid, said di, tri or polyalkyl ester having a formula of 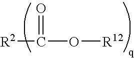
wherein
R² is as previously defined and preferably saturated having 1-24 carbon atoms or unsaturated having 2-24 carbon atoms, R¹² is C₁-C₈ alkyl, such as methyl, ethyl, propyl or butyl, and q is an integer and at least 2,
while removing by-product R¹²OH, and (ii) subjecting the reaction product obtained in Step (i) to aminolysis with at least one alkanolamine while removing by-product R¹²OH.

Suitable reaction temperatures for the alcoholysis as well as the aminolysis are normally but not exclusively found within the range of 150-250° C., such as 160-220° C.

Preferred embodiments of the process according to the present invention include subjecting a di, tri or polyalcohol selected from the group consisting of 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol, to said alcoholysis with a di, tri or polyalkyl ester of a di, tri or polyfunctional carboxylic acid selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid. Alkyl is here suitably linear or branched alkanyl having 1-18 carbon atoms or linear or branched alkenyl having 3-18 carbon atoms and alkoxy and hydroxyalkoxy is preferably derived from at least one alkylene oxide, such as ethylene oxide, propylene oxide and/or hutylene oxide. Said di, tri or polyalcohol is suitably exemplified by compounds such as 2-methyl-1,3-propanediol, 2-methyl-2-ethyl-1,3-propanediol, 2-ethyl-2-butyl-1,3-propanediol, neopentyl glycol, dimethylolpropane, trimethylolethane, trimethylolpropane, di(trimethylolethane), di(trimethylolpropane), pentaerythritol or dipentaerythritol.

Further suitable embodiments of the di, tri or polyalcohol include for instance di, tri or poly(hydroxy)carboxylic acids, wherein the carboxyl group or groups is/are protected using methods as previously disclosed. The most preferred embodiments of said di, tri and poly(hydroxy)carboxylic acid include 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)malonic acid and gluconic acid.

The alkanolamine of Step (ii) is in preferred embodiments of the process selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclohexanol, 2-aminocyclopentanol, 2-amino-2-methyl-1,3-propanediol, 2-amino-2-ethyl-1,3-propanediol and 2-amino-2-hydroxymethyl-1,3-propanediol.

As used throughout this document, R² may be one of the following: 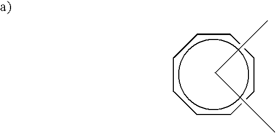
optionally substituted by at least one member selected from the group consisting of alkyl and aryl groups, and optionally fused to one or more aryl groups; 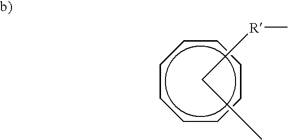
optionally substituted by at least one member selected from the group consisting of alkyl and aryl groups, and optionally fused to one or more aryl groups; 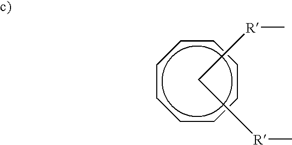
optionally substituted by at least one member selected from the group consisting of alkyl and aryl groups, and optionally fused an aryl group; and
d) R′, optionally substituted by at least one member selected from the group consisting of alkyl and aryl groups;
wherein R′ is an alkanylene or alkenylene group, such as —CₙH₂ₙ—, —CₙH₂ₙ—C₂H₂—, —C₂H₂—CₙH₂ₙ—, or —CₙH₂ₙ—C₂H₂—CₘH₂ₘ—, wherein n and m are independent integers and each at least 1. Similarly, it is understood that terms where terms such as “alkyl”, “alkylaryl” and “arylalkyl” are used, such terms refer only to the relationships between the carbon atoms, and hydrogen atoms may be added or removed such that the particular group may bond to the remainder of the molecule with the correct valences, while simultaneously maintaining the type and number of carbon-to-carbon bonds. Additionally, it is understood that when a particular formula, either by name of by structure is given, it is within the scope of the invention to substitute an isomer thereof in place of the identified formula.

Additionally, R² may be any bivalent straight chain or branched, linear or cyclic, saturated or unsaturated aliphatic hydrocarbon radical, as well as any bivalent aromatic radical, optionally fused to or substituted by another aromatic group or any straight chain or branched, linear or cyclic, saturated or unsaturated aliphatic hydrocarbon group. For example, in one embodiment, R² may comprise any aromatic group, such as —R²⁰R²¹R²²—, wherein R²⁰ and R²² are each independently straight chain or branched, linear or cyclic, saturated or unsaturated aliphatic hydrocarbon radical, and R²¹ is a bivalent aromatic radical.

Without further elaboration, it is believed that one skilled in the art can, using the preceding description, utilize the present invention to its fullest extent. The following preferred specific embodiments are, therefore, to be construed as merely illustrative and not limitative of the remainder of the disclosure in any way whatsoever. It will be understood, of course, that the invention is not limited thereto since many modifications may be made, and it is, therefore, contemplated to cover by the appended claims any such modifications as fall within the true spirit and scope of the invention. In the following Examples 1-8 refer to preparation of embodiments of the β-hydroxyamide and to embodiments of the process of the present invention. Examples 9 and refer to preparation and evaluation of a coating composition comprising β-hydroxyamides according to embodiments of the present invention.

### EXAMPLE 1

Step (i): 765.6 g of dimethyladipate was charged in a reaction flask equipped with electrical heating, a Dean-Stark separator, a vertical cooler, mechanical stirrer and nitrogen inlet. 0.1 g dibutyltinlaurate was under stirring and nitrogen blanket added. The temperature was now raised to 165° C. and 136 g of pentaerythritol was added in small portions while maintaining the temperature and progressively distilling off formed methanol. The temperature increased to 220° C. and the alcoholysis was considered completed when 128 g of methanol was collected. Vacuum was applied and excess of dimethyladipate was evaporated from formed pentaerythritol tetra(methyladipate). The temperature was subsequently decreased to 180° C.

Step (ii) 420 g of diethanolamine was charged at said 180° C. The temperature was subsequently raised to 220° C. and methanol removed from the reaction mixture. The aminolysis was considered completed when 128 g of methanol was collected and vacuum was applied to remove unreacted diethanolamine.

Obtained β-hydroxyamide (1000 g) had a hydroxyl value of 8 mequiv/g.

### EXAMPLE 2

Step (i) 800 g: of Estasol (mixed dibasic ester comprising 21%-w/w of dimethyladipate, 59%-w/w of dimethylglutarate and 20%-w/w of dimethylsuccinate), 193 g of dipentaerythritol, 3 g of dibutyltinoxide and 2 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised to 170° C. and formed methanol progressively distilled off. The temperature was maintained at 170° C. and the alcoholysis was considered completed when 140 ml of methanol was collected. Vacuum was applied and excess of Estasol was evaporated from formed dipentaerythritol ester.

Step (ii): 200 g of the dipentaerythritol ester obtained in Step (i), 124 g of diethanolamine and 0.3 g of dibutyltinoxide were charged in a reaction flask equipped as in Step (i). The temperature was slowly raised to 220° C. and methanol removed from the reaction mixture. The aminolysis was considered completed when 37 ml of methanol was collected. Vacuum was now applied to remove unreacted diethanolamine and obtained β-hydroxylamide was recovered.

Obtained β-hydroxyamide had a hydroxyl value of 8.23 mequiv/g and a viscosity of 740 mPas at 100° C.

### EXAMPLE 3

Step (i): 500 g of Estasol (mixed dibasic ester comprising 21%-w/w of dimethyladipate, 59%-w/w of dimethylglutarate and 20%-w/w of dimethylsuccinate), 106.5 g of pentaerythritol, 1.8 g of dibutyltinoxide and 1.2 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised to 185° C. and formed methanol progressively distilled off. The temperature was allowed to decrease to 165° C. during the alcoholysis and the alcoholysis was considered completed when 100 ml of methanol was collected and unreacted Estasol was distilled off from formed pentaerythritol ester.

Step (ii): 200 g of the pentaerythritol ester obtained in Step (i), 130 g of diethanolamine and 0.3 g of dibutyltinoxide were charged in a reaction flask equipped as in Step (i). The temperature was slowly raised to 220° C. and methanol removed from the reaction mixture. The aminolysis was considered completed when 39 ml of methanol was collected. Vacuum was now applied to remove unreacted diethanolamine and obtained p-hydroxyamide was recovered.

Obtained β-hydroxyamide had a hydroxyl value of 8.52 mequiv/g and a viscosity of 660 mPas at 100° C.

### EXAMPLE 4

Step (i): 500 g of Estasol (mixed dibasic ester comprising 21%-w/w of dimethyladipate, 59%-w/w of dimethylglutarate and 20%-w/w of dimethylsuccinate), 163 g of neopentyl glycol, 2 g of dibutyltinoxide and 1.3 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised the melting point of neopentyl glycol and formed methanol progressively distilled off. The temperature was allowed to decrease 5-10° C. during the alcoholysis and the alcoholysis was considered completed when 100 ml of methanol was collected and unreacted Estasol was distilled off from formed neopenyl glycol ester.

Step (ii): 200 g of the neopentyl glycol ester obtained in Step (i), 117 g of diethanolamine and 0.3 g of dibutyltinoxide were charged in a reaction flask equipped as in Step (i). The temperature was slowly raised to 220° C. and methanol removed from the reaction mixture. The aminolysis was considered completed when 35 ml of methanol was collected. Vacuum was now applied to remove unreacted diethanolamine and obtained β-hydroxyamide was recovered.

Obtained p-hydroxyamide had a hydroxyl value of 7.91 mequiv/g and a viscosity of 260 mPas at 100° C.

### EXAMPLE 5

Step (i): 500 g of Estasol (mixed dibasic ester comprising 21%-w/w of dimethyladipate, 59%-w/w of dimethylglutarate and 20%-w/w of dimethylsuccinate), 210 g of dimethylolpropionic acid, 2.1 g of dibutyltinoxide and 1.4 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised until all dimethylolpropionic acid was dissolved and formed methanol progressively distilled off. The temperature was allowed to decrease 5-10° C. during the alcoholysis and the alcoholysis was considered completed when 100 ml of methanol was collected and unreacted Estasol was distilled off from formed dimethylolpropionic acid ester.

Step (ii): 200 g of the dimethylolpropionic acid ester obtained in Step (i), 108 g of diethanolamine and 0.3 g dibutyltinoxide were charged in a reaction flask equipped as in Step (i). The temperature was slowly raised to 220° C. and methanol removed from the reaction mixture. The aminolysis was considered completed when 33 ml of methanol was collected. Vacuum was now applied to remove unreacted diethanolamine and obtained p-hydroxylamide was recovered.

Obtained β-hydroxyamide had a hydroxyl value of 7.47 mequiv/g and a viscosity of 840 mPas at 100° C.

### EXAMPLE 6

Step (i): 500 g of Estasol (mixed dibasic ester comprising 21%-w/w of dimethyladipate, 59%-w/w of dimethylglutarate and 20%-w/w of dimethylsuccinate), 141 g of 2-methyl-1,3-propanediol, 2 g of dibutyltinoxide and 1.3 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised to 140° C. and formed methanol progressively distilled off. The temperature was further raised to 160° C. and allowed to decrease 5-10° C. during the alcoholysis. The alcoholysis was considered completed when 100 ml of methanol was collected and unreacted Estasol was distilled off from formed 2-methyl-1,3-propanediol ester.

Step (ii): 200 g of the 2-methyl-1,3-propanediol ester obtained in Step (i), 122 g of diethanolamine and 0.3 g of dibutyltinoxide were charged in a reaction flask equipped as in Step (i). The temperature was slowly raised to 220° C. and methanol removed from the reaction mixture. The aminolysis was considered completed when 37 ml of methanol was collected. Vacuum was now applied to remove unreacted diethanolamine and obtained p-hydroxyamide was recovered.

Obtained β-hydroxyamide had a hydroxyl value of 8.13 mequiv/g and a viscosity of 457 mPas at 100° C.

### EXAMPLE 7

Step (i): 800 g of Estasol (mixed dibasic ester comprising 21%-w/w of dimethyladipate, 59%-w/w of dimethylglutarate and 20%-w/w of dimethylsuccinate), 309.5 g of di(trimethylolpropane), 3.3 g of dibutyltinoxide and 2.2 g of trisnonylphenylphosphite were charged in a reaction flask equipped with electrical heating, a Dean-Stark separator, a vertical cooler, mechanical stirrer and nitrogen inlet. The temperature was under stirring and nitrogen blanket raised until all di(trimethylolpropane) was dissolved and formed methanol progressively distilled off. The temperature was allowed to decrease 5-10° C. during the alcoholysis, The alcoholysis was considered completed when 160 ml of methanol was collected and unreacted Estasol was distilled off from formed di(trimethylolpropane) ester.

Step (ii): 200 g of the di(trimethylolpropane) ester obtained in Step (i), 111 g of diethanolamine and 0.3 g of dibutyltinoxide were charged in a reaction flask equipped as in Step (i). The temperature was slowly raised to 220° C. and methanol removed from the reaction mixture. The aminolysis was considered completed when 34 ml of methanol was collected. Vacuum was now applied to remove unreacted diethanolamine and obtained p-hydroxyamide was recovered.

Obtained β-hydroxyamide had a hydroxyl value of 7.62 mequiv/g and a viscosity of 428 mPas at 100° C.

### EXAMPLE 8

Step (i): 500 g of Estasol (mixed dibasic ester comprising 21%-w/w of dimethyladipate, 59%-w/w of dimethylglutarate and 20%-w/w of dimethylsuccinate), 140 g of trimethylolpropane and 2 g of dibutyltinoxide were charged in a reaction flask equipped with electrical heating, a Dean-Stark separator, a vertical cooler, mechanical stirrer and nitrogen inlet. The temperature was under stirring and nitrogen blanket raised to 160° C. and formed methanol progressively distilled off. The temperature was further raised to 200° C. and allowed to decrease 5-10° C. during the alcoholysis. The alcoholysis was considered completed when 100 ml of methanol was collected and unreacted Estasol was distilled off from formed trimethylolpropane ester.

Step (ii): 200 g of the trimethylolpropane ester obtained in Step (i), 122 g of diethanolamine and 0.3 g of dibutyltinoxide were charged in a reaction flask equipped as in Step (i). The temperature slowly raised to 220° C. and methanol removed from the reaction mixture. The aminolysis was considered completed when 37 ml of methanol was collected. Vacuum was now applied to remove unreacted diethanolamine and obtained β-hydroxylamide was recovered.

Obtained β-hydroxyamide had a hydroxyl value of 8.5 mequiv/g and a viscosity of 820 mPas at 100° C.

### EXAMPLE 9

Preparation and evaluation of a coating composition comprising a β-hydroxyamide according to an embodiment of the present invention.

A lacquer was prepared by mixing 1 g of the β-hydroxylamide obtained in Example 1 with a polyurethane dispersion having the following composition:

| | |
|---|---|
| N-methylpyrrolidone: | 7.37 g |
| isophorone diisocyanate: | 11,61 g |
| dimethylolpropionic acid: | 1.50 g |
| polypropylene glycol adipate: | 11.67 g |
| trimethylolpropane: | 0.14 g |
| dimethylethanolamine: | 1.30 g |
| ethylenediamine: | 1.02 g |
| water: | 56.00 g |

0.20 g of a silicon defoamer was added and 0.50 g a dimethylsiloxane based substrate wetting agent was finally mixed into obtained lacquer composition. The lacquer, a milky waterborne liquid, was applied on a glass substrate at a thickness of 120 μm wet film and cured at 150° C. to yield a clear coating having a König hardness of 100 oscillations.

### EXAMPLE 10

Preparation and evaluation of a coating composition comprising a β-hydroxylamide according to an embodiment of the present invention.

A polyester was prepared, according to a standard procedure, from 2 moles of endomethylene tetrahydrophthalic anhydride and 1 mole of pentaerythritol. Obtained polyester had an acid value of 95-110 mg KOH/g and was diluted to a non-volatile content of 75% by weight in xylene.

A lacquer was prepared by mixing 100 g said polyester with 21 g of the β-hydroxylamide obtained in Example 8. The lacquer was applied on a glass substrate at a thickness of 150 μm wet film and cured at 200° C. to yield a clear coating having a König hardness of 150 oscillations.

1. A β-hydroxyamide having a general formula of 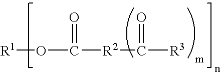
wherein
R¹ is a linear, branched or cyclic saturated or unsaturated aliphatic group, which group optionally comprises one or more ether and/or ester units,
R² is a linear, branched or cyclic, saturated or unsaturated, aliphatic and/or aromatic group,
R³ is N-alkyl or N-cycloalkyl having at least one hydroxyl group in P-position, m is an integer and at least 1 and n is an integer and at least 2.

2. A β-hydroxyamide according to claim 1, wherein said β-hydroxyamide is obtained by subjecting a di, tri or polyalcohol to alcoholysis with a di, tri or polyalkyl ester of a di, tri or polyfunctional carboxylic acid and by subsequently subjecting obtained reaction product to aminolysis with at least one alkanolamine.

3. A β-hydroxyamide according to claim 1, wherein, R¹ is derived from at least one compound having at least two hydroxyl groups.

4. A β-hydroxyamide according to claim 1, wherein R² is derived from at least one compound having at least two carboxyl groups or from an anhydride, a halide or an alkyl ester or ether of a compound having said at least two carboxyl groups.

5. A β-hydroxyamide according to claim 1, wherein R³ is derived from at least one alkanolamine.

6. A β-hydroxyamide according to claim 1, wherein R¹ is a saturated group having 1-24 carbon atoms.

7. A β-hydroxyamide according to claim 1, wherein R¹ is an unsaturated group having 2-24 carbon atoms.

8. A β-hydroxyamide according to claim 1, wherein R² is a group having 1-24 carbon atoms.

9. A β-hydroxyamide according to claim 1, wherein R² is a group having 2-24 carbon atoms.

10. A β-hydroxyamide according to claim 1, wherein said N-alkyl is N-alkanyl having 2-20 carbon atoms and said N-cycloalkyl is N-cycloalkanyl having 3-20 carbon atoms.

11. A β-hydroxyamide according to claim 1, wherein said one or more ether units of R¹ is/are derived from at least one alkylene oxide.

12. A β-hydroxyamide according to claim 11, wherein said alkylene oxide is at least one selected from the group consisting of ethylene oxide, propylene oxide and butylene oxide.

13. A β-hydroxyamide according to claim 1, wherein R³ is a group of formula 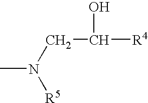
wherein R⁴ is alkyl and R⁵ is hydrogen or a group of formula 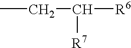
wherein R⁶ is alkyl and R⁷ is selected from the group consisting of hydrogen, hydroxyl, saturated or unsaturated aliphatic group and hydroxyalkyl.

14. A β-hydroxylamide according to claim 13, wherein said aliphatic group is a linear or branched alkanyl having 1-20 carbon atoms.

15. A β-hydroxyamide according to claim 3, wherein said compound having said at least two hydroxyl groups is a di, tri or polyalcohol of formula 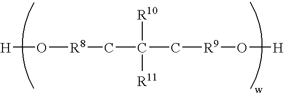
wherein:
w is an integer and at least 1,
R⁸ and R⁹ each independently is a group of formula —(CₓH₂ₓ)_{y}—, —(CᵣH₂ᵣOₚ₋₁)ₚ— or —(CₓH₂ₓ)_{y}(CᵣH₂ᵣOₚ₋₁)ₚ—, and
R¹⁰ and R¹¹ each independently is H, —OH, —COOH or a group of formula —(CₓH₂ₓ₊₁)_{y}, —(CₓH₂ₓ)_{y}OH, —(CₓH₂ₓ)_{y}COOH, —(CᵣH₂ᵣO)ₚH, —(CₓH₂ₓ)_{y}(CᵣH₂ᵣO)ₚH,
wherein x, y, r and p are independent integers being at least 1.

16. A β-hydroxyamide according to claim 15, wherein said di, tri or polyalcohol is selected from the group consisting of a 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol.

17. A β-hydroxyamide according to claim 16, wherein said alkyl is a linear or branched alkanyl having 1-18 carbon atoms or a linear or branched alkenyl having 3-18 carbon atoms.

18. A β-hydroxyamide according to claim 16, wherein said alkoxy and/or said hydroxyalkoxy is derived from at least one alkylene oxide.

19. A β-hydroxyamide according to claim 18, wherein said alkylene oxide is ethylene oxide, propylene oxide and/or butylene oxide.

20. A β-hydroxyamide according to claim 16, wherein said di, tri or polyalcohol is 2-methyl-1,3-propanediol, 2-methyl-2-ethyl-1,3-propanediol, 2-ethyl-2-butyl-1,3-propanediol, neopentyl glycol, dimethylolpropane, trimethylolethane, trimethylolpropane, di(trimethylolethane), di(trimethylolpropane), pentaerythritol or dipentaerythritol.

21. A β-hydroxyamide according to claim 3, wherein said compound having said at least two hydroxyl groups is a monoallyl or mono(methallyl) ether of glycerol, trimethylolethane, trimethylolpropane, di(trimethylolethane), di(trimethylolpropane) or pentaerythritol.

22. A β-hydroxylamide according to claim 3, wherein said compound having said at least two hydroxyl groups is a diallyl or di(methallyl) ether of di(trimethylolethane), di(trimethylolpropane) or pentaerythritol.

23. A β-hydroxylamide according to claim 3, wherein said compound having said at least two hydroxyl groups is glycerol, diglycerol, anhydroenneaheptitol, sorbitol or mannitol.

24. A β-hydroxyamide according to claim 3, wherein said compound having said at least two hydroxyl groups is a di, tri, or poly(hydroxy)carboxylic acid, wherein carboxyl group or groups optionally is/are protected.

25. A β-hydroxyamide according to claim 24, wherein said di, tri, or poly(hydroxy)carboxylic acid is at least one selected from the group consisting of 2,2-dimethylolpropionic acid, α,β-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di (hydroxy)benzoic acid, α,β-di (hydroxy)propionic acid, heptonic acid, citric acid, tartaric acid, di(hydroxy)malonic acid and gluconic acid.

26. A β-hydroxyamide according to claim 1, wherein R² is derived from a di, tri or polyfunctional carboxylic acid, from an anhydride of a di, tri or polyfunctional carboxylic acid or from a di, tri or polyalkyl ester of a di, tri or polyfunctional carboxylic acid.

27. A β-hydroxyamide according to claim 26, wherein said di, tri or polyfunctional carboxylic acid is at least one selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid.

28. A β-hydroxyamide according to claim 2, wherein said alkanolamine is at least one selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclo-hexanol, 2-aminocyclopentanol, 2-amino-2-methyl-1,3-propanediol, 2-amino-2-ethyl-1,3-propanediol and 2-amino-2-hydroxymethyl-1,3-propanediol.

29. A process for synthesis of a β-hydroxyamide according to claim 1, wherein said process comprises the steps of:
i) subjecting a di, tri or polyalcohol of formula 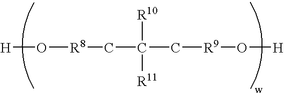
wherein w is an integer and at least 1, R⁸ and R⁹ each independently is a group of formula —(CₓH₂ₓ)_{y}—, —(CᵣH₂ᵣOₚ₋₁)ₚ— or —(CₓH₂ₓ)_{y}(CₓH₂ᵣOₚ₋₁)ₚ—, and R¹⁰ and R¹¹ each independently is —H, —OH, —COOH or a group of formula —(CₓH₂₊₁)_{y}, —(CₓH₂ₓ)_{y}OH, —(CₓH₂ₓ)_{y}COOH, —(CᵣH₂ᵣO)ₚH, —(CₓH₂ₓ)_{y}(CᵣH₂ᵣO)ₚH, wherein x, y, r and p are independent integers being at least 1,
to alcoholysis with a di, tri or polyalkyl ester of a di, tri or polyfunctional carboxylic acid, said di, tri or polyalkyl ester having a formula of 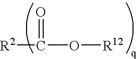
wherein R² is a linear, branched or cyclic saturated or unsaturated aliphatic group, which
group optionally comprises one or more ether and/or ester, R¹² is C₁-C₈ alkyl, and q is an interger and at least 2, while removing by-product R¹²OH, and
ii) subjecting the product of step i) to aminolysis with at least one alkanolamine, while removing by-product R¹²OH.

30. A process according to claim 29, wherein R² is selected from the group consisting of alkenyl, aryleneyl, alkenyl, arylenealkyl, aryl, alkylaryl and arylalkyl groups, wherein alkyl is linear or branched alkanyl having 1-24 carbon atoms or linear or branched alkenyl having 2-24 carbons.

31. A process according to claim 29, wherein R¹² is methyl, ethyl, propyl or butyl.

32. A process according to claim 29, wherein said di, tri or polyalcohol is at least one selected from the group consisting of a 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol.

33. A process according claim 32, wherein said alkyl is linear or branched alkanyl having 1-18 carbon atoms or linear or branched alkenyl having 3-18 carbon atoms.

34. A process according to claim 32, wherein said alkoxy and/or said hydroxyalkoxy is derived from at least one alkylene oxide.

35. A process according to claim 34, wherein said alkylene oxide is at least one selected from the group consisting of ethylene oxide, propylene oxide and butylene oxide.

36. A process according to claim 29, wherein said di, tri or polyalcohol is at least one selected from the group consisting of 2-methyl-1,3-propanediol, 2-methyl-2-ethyl-1,3-propanediol, 2-ethyl-2-butyl-1,3-propanediol, neopentyl glycol, dimethylolpropane, trimethylolethane, trimethylolpropane, di(trimethylolethane), di(trimethylolpropane), pentaerythritol and dipentaerythritol.

37. A process according to claim 29, wherein said di, tri or polyalcohol is a di, tri or poly(hydroxy)carboxylic acid, wherein the carboxyl group or groups optionally is/are protected.

38. A process according to claim 37, wherein said di, tri, or poly(hydroxy)carboxylic acid is selected from the group consisting of 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)malonic acid and gluconic acid.

39. A process according to claim 29, wherein said di, tri or polyfunctional carboxylic acid is at least one selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid.

40. A process according to claim 29, wherein said alkanolamine is at least one selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclo-hexanol, 2-aminocyclopentanol, 2-amino-2-methyl-1,3-propanediol, 2-amino-2-ethyl-1,3-propanediol and 2-amino-2-hydroxymethyl-1,3-propanediol.

41. A process according to claim 29, wherein said alcoholysis is performed at a temperature of 150-250° C.

42. A process according to claim 29, wherein said aminolysis is performed at a temperature of 150-250° C.

43. A process according to claim 29, wherein said alcoholysis is performed at a temperature of 160-220° C.

44. A process according to claim 29, wherein said aminolysis is performed at a temperature of 180-220° C.

45. A β-hydroxylamide according to claim 29, wherein R² is an unsaturated aliphatic group.

46. A β-hydroxyamide wherein it has a general formula of 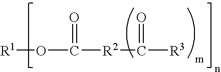
wherein
R¹ is alkylene or alkoxyalkylene,
R² is alkylene, arylene, alkylarylene, or arylalkylene;
R³ is N-alkyl or N-cycloalkyl having at least one hydroxyl group in P-position, m is an integer and at least 1 and wherein n is an integer and at least 2.

47. A β-hydroxyamide according to claim 46, wherein it is obtained by subjecting a di, tri or polyalcohol to alcoholysis with a di, tri or polyalkyl ester of a di, tri or polyfunctional carboxylic acid and by subsequently subjecting obtained reaction product to aminolysis with at least one alkanolamine.

48. A β-hydroxyamide according to claim 46, wherein R¹ is a group derived from at least one compound having at least two hydroxyl groups.

49. A β-hydroxyamide according to claim 46, wherein R² is a group derived from at least one compound having at least two carboxyl groups or from an anhydride, a halide or an alkyl ester or ether of a compound having said at least two carboxyl groups.

50. A β-hydroxyamide according to claim 46, wherein R³ is a group derived from at least one alkanolamine.

51. A β-hydroxyamide according to claim 46, wherein R² comprises at least one ester and/or ether group.

52. A β-hydroxyamide according to claim 46, wherein R² is linear or branched alkanylene or alkenylene.

53. A β-hydroxyamide according to claim 46, wherein R² has 1-24 carbon atoms.

54. A β-hydroxyamide according to claim 46, wherein said N-alkyl is N-alkanyl having 2-20 carbon atoms and said N-cycloalkyl is N-cycloalkanyl having 3-20 carbon atoms.

55. A β-hydroxyamide according to claim 46, wherein alkoxy in said alkoxyalkyl is
—(CᵣH₂ᵣO)ₚ— wherein r and p are independent integers being at least 1.

56. A β-hydroxyamide according to claim 46, wherein alkoxy in said alkoxyalkyl is derived from at least one alkylene oxide.

57. A β-hydroxyamide according to claim 56, wherein said alkylene oxide is ethylene oxide, propylene oxide and/or butylene oxide.

58. A β-hydroxyamide according to claim 46, wherein R³ is a group of formula 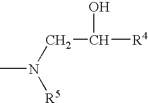
wherein R⁴ is alkyl and R⁵ is hydrogen or a group of formula 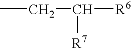
wherein R⁶ is alkyl and R⁷ is hydrogen, hydroxyl, alkyl or hydroxyalkyl.

59. A β-hydroxyamide according to claim 58, wherein said alkyl is linear or branched, having 1-20 carbon atoms.

60. A β-hydroxyamide according to claim 48, wherein said compound having said at least two hydroxyl groups is a di, tri or polyalcohol of formula 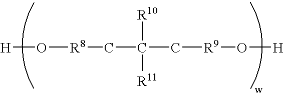
wherein w is an integer and at least 1, R⁸ and R⁹ each independently is a group of formula —(CₓH₂ₓ)_{y}—, —(CᵣH₂ᵣOₚ₋₁)ₚ— or —(CₓH₂ₓ)_{y}(CᵣH₂ᵣOₚ₋₁)ₚ—, and R¹⁰ and R¹¹ each independently is —H, —OH, —COOH or a group of formula —(CₓH₂ₓ₊₁)_{y}, —(CₓH₂ₓ)_{y}OH, —(CₓH₂ₓ)_{y}COOH, (CᵣH₂ᵣO)ₚH, —(CₓH₂ₓ)_{y}(CᵣH₂ᵣO)ₚH, wherein x, y, r and p are independent integers being at least 1.

61. A β-hydroxyamide according to claim 60, wherein said di, tri or polyalcohol is selected from the group consisting of a 2-alkyl-1,3-propanediol, a 2,2-dialkyl-1,3-propanediol, a 2-hydroxy-1,3-propanediol, a 2,2-dihydroxy-1,3-propanediol,
a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol.

62. A β-hydroxyamide according to claim 61, wherein R² is linear or branched alkanyl having 1-18 carbon atoms or linear or branched alkenyl having 3-18 carbon atoms.

63. A β-hydroxyamide according to claim 61, wherein said alkoxy and/or said hydroxyalkoxy is derived from at least one alkylene oxide.

64. A β-hydroxyamide according to claim 63, wherein said alkylene oxide is ethylene oxide, propylene oxide and/or butylene oxide.

65. A β-hydroxyamide according to claim 63, wherein said di, tri or polyalcohol is 2-methyl-1,3-propanediol, 2-methyl-2-ethyl-1,3-propanediol, 2-ethyl-2-butyl-1,3-propanediol, neopentyl glycol, dimethylolpropane, trimethylolethane, trimethylolpropane, di(trimethylolethane), di(trimethylolpropane), pentaerythritol or dipentaerythritol.

66. A β-hydroxyamide according to claim 48, wherein said compound having said at least two hydroxyl groups is a monoallyl or mono(methallyl) ether of glycerol, trimethylolethane, trimethylolpropane, di(trimethylolethane), di(trimethylolpropane) or pentaerythritol.

67. A β-hydroxyamide according to claim 48, wherein said compound having said at least two hydroxyl groups is a diallyl or di(methallyl) ether of di(trimethylolethane), di(trimethylolpropane) or pentaerythritol.

68. A β-hyroxyamide according to claim 48, wherein said compound having said at least two hydroxyl groups is glycerol, diglycerol, anhydroenneaheptitol, sorbitol, or mannitol.

69. A β-hydroxyamide according to claim 48, wherein said compound having said at least two hydroxyl groups is a di, tri, or poly(hydroxy)carboxylic acid, wherein the carboxyl group or groups optionally is/are protected.

70. A β-hydroxyamide according to claim 69, wherein said di, tri, or poly(hydroxy)carboxylic acid is 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)malonic acid and/or gluconic acid.

71. A β-hydroxyamide according to claim 46, wherein R² is derived from a di, tri or polyfunctional carboxylic acid, from an anhydride of a di, tri or polyfunctional carboxylic acid or from a di, tri or polyalkyl ester of a di, tri or polyfunctional carboxylic acid.

72. A β-hydroxyamide according to claim 71, wherein said alkyl is C₁-C₁₈.

73. A β-hydroxyamide according to claim 71, wherein said di, tri or polyfunctional carboxylic acid is selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid.

74. A β-hydroxyamide according to claim 73, wherein said alkanolamine is selected from the group consisting of monoethanolamine, diethanolamine, mono-n-propanolamine, di-n-propanolamine, monoisopropanolamine, diisopropanolamine, mono-n-butanolamine, di-n-butanolamine, monoisobutanolamine, diisobutanolamine, mono-sec-butanolamine, di-sec-butanolamine, methylethanolamine, n-butylethanolamine, isobutylethanolamine, N-acetylethanolamine, 2-aminocyclo-hexanol, 2-aminocyclopentanol, 2-amino-2-methyl-1,3-propanediol, 2-amino-2-ethyl-1,3-propanediol and 2-amino-2-hydroxymethyl-1,3-propanediol.

75. A process for synthesis of a β-hydroxyamide according to claim 46, wherein said process comprises the steps of
i) subjecting a di, tri or polyalcohol of formula 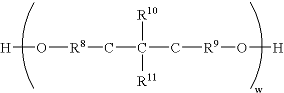
wherein w is an integer and at least 1, R⁸ and R⁹ each independently is a group of formula —(CₓH₂ₓ)_{y}—, (CᵣH₂ᵣOₚ₋₁)ₚ— or —(CₓH₂ₓ)_{y}(CᵣH₂ᵣOₚ₋₁)ₚ—, and R¹⁰ and R¹¹ each independently is —H, —OH, —COOH or a group of formula —(CₓH₂ₓ₊₁)_{y}, —CH₂ₓ)_{y}OH, —(CₓH₂ₓ)_{y}COOH, —(CᵣH₂ᵣO)ₚH, —(CₓH₂ₓ)_{y}(CᵣH₂ᵣO)ₚH, wherein x, y, r and p are independent integers being at least 1,
to alcoholysis with a di, tri or polyalkyl ester of a di, tri or polyfunctional carboxylic acid, said di, tri or polyalkyl ester having a formula of 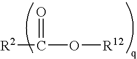
wherein R² is alkylene, arylene, alkylarylene, or arylalkylene, wherein said alkylene is linear or branched alkanylene having 1-24 carbon atoms or linear or branched alkenylene having 2-24 carbon atoms, R¹² is C₁-C₈ alkyl, and q is an integer and at least 2,
while removing by-product R¹²OH, and
ii) subjecting in Step (i) obtained reaction product to aminolysis with at least one alkanolamine while removing by-product R¹²OH.

76. A process according to claim 75, wherein R¹² is methyl, ethyl, propyl or butyl.

77. A process according to claim 75, wherein said di, tri or polyalcohol is selected from the group consisting of 2-alkyl-1,3-propanediol, a 2,2-dialkyl-1,3-propanediol, a 2-hydroxy-1,3-propanediol, a 2,2-dihydroxy-1,3-propanediol, a 2-hydroxy-2-alkyl-1,3-propanediol, a 2-hydroxyalkyl-2-alkyl-1,3-propanediol, a 2,2-di(hydroxyalkyl)-1,3-propanediol, a 2-hydroxyalkoxy-2-alkyl-1,3-propanediol, a 2,2-di(hydroxyalkoxy)-1,3-propanediol, a 2-hydroxyalkoxyalkyl-2-alkyl-1,3-propanediol, a 2,2-di(hydroxyalkoxyalkyl)-1,3-propanediol and a dimer, trimer or polymer of a said 1,3-propanediol.

78. A process according to claim 77, wherein said alkyl is linear or branched alkanyl having 1-18 carbon atoms or linear or branched alkenyl having 3-18 carbon atoms.

79. A process according to claim 77, wherein said alkoxy and/or said hydroxyalkoxy is derived from at least one alkylene oxide.

80. A process according to claim 79, wherein said alkylene oxide is ethylene oxide, propylene oxide and/or butylene oxide.

81. A process according to claim 75, wherein said di, tri or polyalcohol is 2-methyl-1,3-propanediol, 2-methyl-2-ethyl-1,3-propanediol, 2-ethyl-2-butyl-1,3-propanediol, neopentyl glycol, dimethylolpropane, trimethylolethane, trimethylolpropane, di(trimethylolethane), di(trimethylolpropane), pentaerythritol or dipentaerythritol.

82. A process according to claim 81, wherein said di, tri or polyalcohol is a di, tri or poly(hydroxy)carboxylic acid, wherein the carboxyl group or groups optionally is/are protected.

83. A process according to claim 82, wherein said di, tri, or poly(hydroxy)carboxylic acid is 2,2-dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)propionic acid, 3,5-di(hydroxy)benzoic acid, α,β-di(hydroxy)propionic acid, heptonic acid, citric acid, tartaric acid, di(hydroxy)malonic acid and/or gluconic acid.

84. A process according to claim 75, wherein said di, tri or polyfunctional carboxylic acid is selected from the group consisting of adipic acid, azelaic acid, fumaric acid, maleic acid, phthalic acid, isophthalic acid, terephthalic acid, tetrahydrophthalic acid, hexahydrophthalic acid, succinic acid, sebacic acid, diglycolic acid, trimelletic acid, citric acid and pyromelletic acid.

85. A process according to claim 75, wherein said alcoholysis is performed at a temperature of 150-250° C.

86. A process according to claim 75, wherein said aminolysis is performed at a temperature of 150-250° C.

87. A β-hydroxyamide according to claim 71, wherein said alkyl is C₁-C₈ linear or branched alkanyl.

88. A β-hydroxyamide according to claim 71, wherein said alkyl is C₁-C₄ linear or branched alkanyl.

89. A process according to claim 74, wherein said alcoholysis is performed at a temperature of 160-220° C.

90. A process according to claim 74, wherein said alcoholysis is performed at a temperature of 180-220° C.
